(19) United States
(12) Patent Application Publication
(10) Pub. No.: US 2026/0023426 A1
(43) Pub. Date: Jan. 22, 2026

(54) TECHNIQUES FOR MULTIPLE WEARABLE DEVICES

(71) Applicant: Oura Health Oy, Oulu (FI)

(72) Inventors: Yijen Liu, San Francisco, CA (US); Rebecca Plotnick, San Francisco, CA (US); Sally Chan, Brooklyn, NY (US); Chris Beale, Pacifica, CA (US); Simon Ratner, San Francisco, CA (US); Alex Christian Earll, North Bend, WA (US); Krystle de Mesa, Redwood City, CA (US)

(21) Appl. No.: 19/271,587

(22) Filed: Jul. 16, 2025

Related U.S. Application Data
(60) Provisional application No. 63/672,533, filed on Jul. 17, 2024.

(51) Int. Cl. G06F 3/01; A61B 5/00; A61B 5/0205; A61B 5/024; A61B 5/026; A61B 5/08; A61B 5/117

(52) U.S. Cl. CPC G06F 3/011; A61B 5/02055; A61B 5/117; A61B 5/6802; A61B 5/7267; A61B 5/7435; A61B 5/7445; G06F 3/017; A61B 5/02405; A61B 5/0261; A61B 5/0816; A61B 5/4809

(57) ABSTRACT

Methods, systems, and devices for multiple wearable devices are described. A user may implement a system of multiple wearable devices, such as a wearable ring device and wearable smart glasses, to gain additional insights regarding their environment and overall health. The smart glasses may generate an augmented reality (AR) visualization that is overlaid via the lenses of the smart glasses when a wearable smart device (e.g., smart ring) is depicted within a field of view of the smart glasses. For example, a user may be able to glance at their smart ring, and their smart glasses may display (e.g., via the overlaid AR visualization) a “live” heart rate measurement collected by the smart ring. Additionally, data collected from the respective wearable devices may be used as inputs to one another, and may be used to provide additional context that may be leveraged to interpret data collected from the respective devices.

### CROSS REFERENCE

The present application for patent claims the benefit of U.S. Provisional Patent Application No. 63/672,533 by LIU et al., entitled “TECHNIQUES FOR MULTIPLE WEARABLE DEVICES,” filed Jul. 17, 2024, assigned to the assignee hereof, and expressly incorporated by reference herein.

### FIELD OF TECHNOLOGY

The following relates to wearable devices and data processing, including techniques for multiple wearable devices, and using data from one wearable device to adjust/control operation of another wearable device.

### BACKGROUND

Some wearable devices may be configured to collect data from users associated with motion data, temperature data, heart rate data, photoplethysmography (PPG) data, and the like. Some users have a desire for more insights regarding their physiological data and overall health.

### BRIEF DESCRIPTION OF THE DRAWINGS

FIG. 1 illustrates an example of a system that supports techniques for multiple wearable devices in accordance with aspects of the present disclosure.

FIG. 2 illustrates an example of a system that supports techniques for multiple wearable devices in accordance with aspects of the present disclosure.

FIG. 3 shows an example of a wearable system that supports techniques for multiple wearable devices in accordance with aspects of the present disclosure.

FIG. 4 show example wearable use cases that supports techniques for multiple wearable devices in accordance with aspects of the present disclosure

FIG. 5 shows an example of a graphical user interface (GUI) that supports techniques for multiple wearable devices in accordance with aspects of the present disclosure.

FIG. 6 shows a block diagram of an apparatus that supports techniques for multiple wearable devices in accordance with aspects of the present disclosure.

FIG. 7 shows a block diagram of a wearable device manager that supports techniques for multiple wearable devices in accordance with aspects of the present disclosure.

FIG. 8 shows a diagram of a system including a device that supports techniques for multiple wearable devices in accordance with aspects of the present disclosure.

FIGS. 9-11 show flowcharts illustrating methods that support techniques for multiple wearable devices d.

### DETAILED DESCRIPTION

A user may use a device (e.g., a wearable device) to determine physiological measurements of the user, such as motion data, temperature data, heart rate data, photoplethysmography (PPG) data, and the like. Physiological data collected via a wearable device may be used to gain insights into the user's sleeping patterns and overall health. However, the physiological data alone may not provide enough information regarding factors that may be affecting the user's overall health. For example, other factors that may affect a user's physiological data may include the food the user consumes and characteristics of the user's environment and surroundings (e.g., surrounding temperature, noise levels, light levels, etc.). As such, physiological data alone may not provide the entire picture regarding factors that are negatively (or positively) affecting the user's overall health.

Accordingly, aspects of the present disclosure are directed to a wearable system that includes multiple wearable devices, such as a wearable ring device and wearable smart glasses, to provide additional insights regarding the user's environment and overall health. Leveraging data from multiple devices may help provide a more complete picture regarding factors that may affect the user's overall health. For example, images collected via the wearable smart glasses may be used to provide additional context regarding physiological data collected from the wearable ring device. For instance, images of the user's surrounding environment (e.g., images of traffic extending in front of the user, images of a plate of food) may be used to determine why the user's stress has increased, or why their heart rate has changed. In additional or alternative aspects, data collected from the respective wearable devices of the wearable system may be used as inputs to one another. For example, gestures recognized via the wearable ring device may be used to trigger wearable smart glasses to take an image.

Aspects of the disclosure are initially described in the context of systems supporting physiological data collection from users via wearable devices. Additional aspects of the disclosure are described in the context of an example graphical user interface (GUI). Aspects of the disclosure are further illustrated by and described with reference to apparatus diagrams, system diagrams, and flowcharts that relate to techniques for multiple wearable devices.

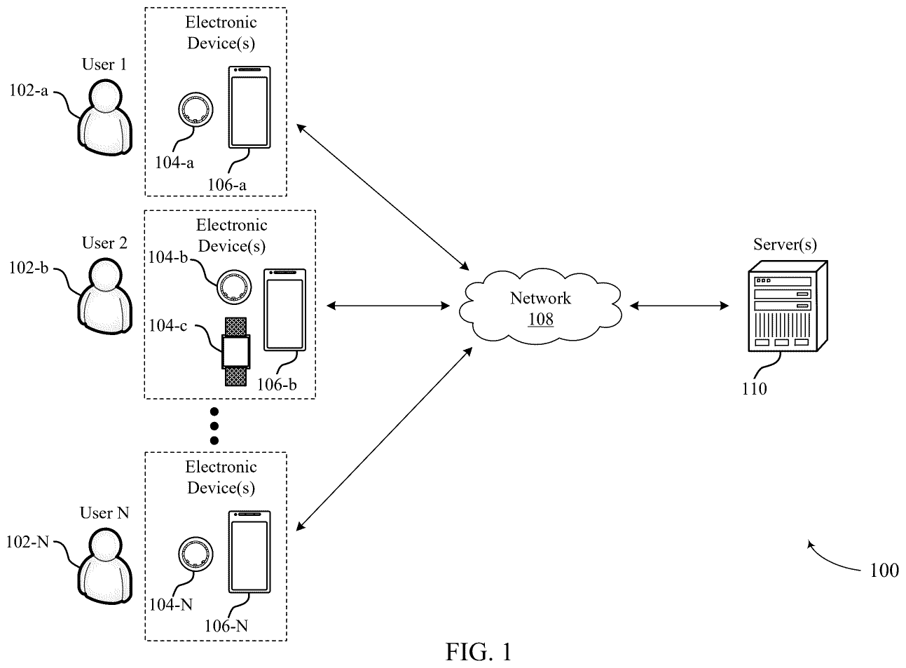

FIG. 1 illustrates an example of a system 100 that supports techniques for multiple wearable devices in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols. Moreover, in some cases, the wearable device 104 and the user device 106 may be included within (or make up) the same device. For example, in some cases, the wearable device 104 may be configured to execute an application associated with the wearable device 104, and may be configured to display data via a GUI.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for a wearable system that includes multiple wearable devices, such as a wearable ring device and wearable smart glasses, to provide additional insights regarding the user's environment and overall health. Leveraging data from multiple devices may help provide a more complete picture regarding factors that may affect the user's overall health. For example, images collected via the wearable smart glasses may be used to provide additional context regarding physiological data collected from the wearable ring device. For instance, images of the user's surrounding environment (e.g., images of traffic extending in front of the user, images of a plate of food) may be used to determine why the user's stress has increased, or why their heart rate has changed. In additional or alternative aspects, data collected from the respective wearable devices of the wearable system may be used as inputs to one another. For example, gestures recognized via the wearable ring device may be used to trigger wearable smart glasses to take an image.

It should be appreciated by a person skilled in the art that one or more aspects of the disclosure may be implemented in a system 100 to additionally, or alternatively, solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

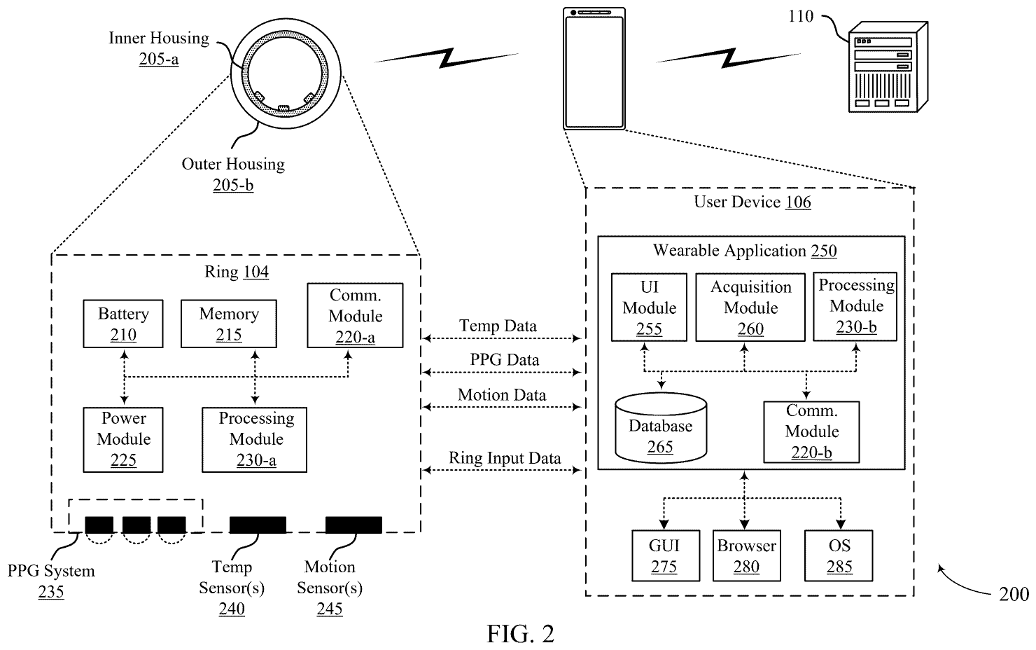

FIG. 2 illustrates an example of a system 200 that supports techniques for multiple wearable devices in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMl160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

In some cases, the wearable device 104 and the user device 106 may be included within (or make up) the same device. For example, in some cases, the wearable device 104 may be configured to execute the wearable application 250, and may be configured to display data via the GUI 275.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, data collected by the wearable device 204, and/or analyses performed by the wearable device 204, the user device 206, and/or the servers 110, may be used to adjust operational parameters of the wearable device 204. For example, based on a determined heart rate of the user and/or a determined activity state of the user, the wearable device 204 may adjust a sampling rate for measuring the user's heart rate, and/or may activate or deactivate certain sensors and/or physiological measurements (e.g., deactivate SpO2 measurements when the user is engaged in physical activity, or otherwise exhibits an activity/movement level above some threshold). By way of another example, the user device 206 and/or the servers 110 may calculate a Readiness Score for the user, and may deactivate or disable activity measurements performed by the wearable device 204 in cases where the Readiness Score is below some threshold (in order to reduce power consumption and conserve battery at the wearable device 204, and/or to disincentivize the user from performing rigorous activity when their Readiness Score is below the threshold value). In this regard, any measurements, calculations, and/or analyses performed by the various devices within the system 200 (e.g., wearable device 204, user device 206, servers 110) may be used by the system 200 to control and/or adjust the operational parameters of the wearable device 204.

Operational parameters that may be controlled/adjusted at the wearable device 204 based on collected data and/or analyses performed by the system 200 may include, but are not limited to, a periodicity/frequency that measurements are performed (e.g., sampling rate), a power level or intensity of LEDs, algorithms used to analyze data at the wearable device 204, what types of measurements are performed (e.g., enabling/disabling specific sensors or types of measurements), a periodicity or frequency that the wearable device 204 transmits data to the user device 206, or any combination thereof. Adjusting operational parameters of the wearable device 204 based on collected data and/or analyses performed by the system 200 may reduce power consumption and improve battery performance at the wearable device 204, and may lead to higher quality data collected by the wearable device 204, thereby enabling the system 200 to perform more accurate and reliable analyses/diagnoses of the user's physiological parameters, and leading to better guidance and insights that may enable the user to improve their overall health.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Duc to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for a wearable system that includes multiple wearable devices, such as a wearable ring device and wearable smart glasses, to provide additional insights regarding the user's environment and overall health. Leveraging data from multiple devices may help provide a more complete picture regarding factors that may affect the user's overall health. For example, images collected via the wearable smart glasses may be used to provide additional context regarding physiological data collected from the wearable ring device. For instance, images of the user's surrounding environment (e.g., images of traffic extending in front of the user, images of a plate of food) may be used to determine why the user's stress has increased, or why their heart rate has changed. In additional or alternative aspects, data collected from the respective wearable devices of the wearable system may be used as inputs to one another. For example, gestures recognized via the wearable ring device may be used to trigger wearable smart glasses to take an image.

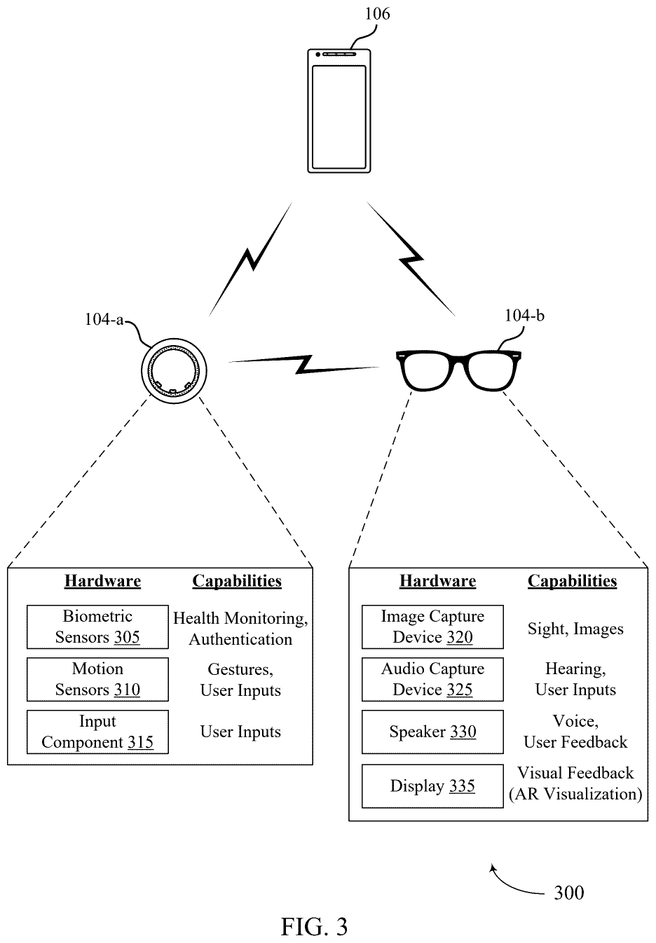

FIG. 3 shows an example of a wearable system 300 that supports techniques for multiple wearable devices in accordance with aspects of the present disclosure. Aspects of the wearable system 300 may implement, or be implemented by, aspects of the system 100, the system 200, or both.

The wearable system 300 may include a user device 106 and multiple wearable devices 104, which may be examples of corresponding devices described herein. In some aspects, the respective wearable devices 104 may be worn at different locations of the user's body. For example, as shown in FIG. 3, the wearable system 300 may include a first wearable device 104-a, which may be an example of a wearable ring device that is configured to be worn on a finger of the user, and a second wearable device 104-b, which may be an example of wearable smart glasses that are configured to be worn on the user's head/face.

As noted previously herein, wearable devices 104 may be configured to collect physiological data from the user, such as motion data, temperature data, heart rate data, PPG data, and the like. Physiological data collected via a wearable device 104 may be used to gain insights into the user's sleeping patterns and overall health. However, the physiological data alone may not provide enough information regarding factors that may be affecting the user's overall health. For example, other factors that may affect a user's physiological data may include the food the user consumes and characteristics of the user's environment and surroundings. As such, physiological data alone may not provide the entire picture regarding factors that are negatively (or positively) affecting the user's overall health.

Accordingly, the wearable system 300 may be configured to leverage data collected from each of the wearable devices 104 in order to gain a more complete picture of the user's overall health. For example, data environmental collected from the second wearable device 104-b (e.g., images and/or audio captured by the wearable smart glasses) may be used to provide additional context for physiological data collected via the first wearable device 104-a, and may be used to identify impacts that the user's environment has on the user's health.

Moreover, in some cases, data collected from the respective wearable devices 104 may be used to adjust parameters of the other device, and/or may otherwise be used as inputs for the other smart device. For example, gestures identified by the first wearable device 104-a may be used to cause the second wearable device 104-b to capture image and/or audio data. By way of another example, if the second wearable device 104-b captures images of a busy street or crowded room, such images may be used to cause the first wearable device 104-a to perform stress-related measurements (e.g., trigger the first wearable device 104-a to perform PPG measurements and/or ECG measurements to determine how the user's environment is affecting their stress levels). Further, images and/or audio collected by the second wearable device 104-b may be used to determine a location of the user (e.g., whether the user is indoors, outdoors, etc.), where the location of the user may be used to adjust when/how the first wearable device 104-a performs measurements (e.g., adjust a measurement periodicity, adjust LED intensity, etc.).

As shown in FIG. 3, the first wearable devices 104-a and the second wearable device 104-b may be directly wirelessly coupled with one another, and/or via the user device 106. In this regard, in some cases, the first wireless device 104-a and the second wearable device 104-b may exchange data and signals with one another. In other cases, the user device 106 may be configured to analyze data received from the respective devices and/or relay data/signals between the respective devices.

In some aspects, the various hardware components of the respective wearable devices 104 may be leveraged to achieve various capabilities that may be used to augment the performance and/or capabilities of the wearable system 300 as a whole. In other words, the data/capabilities associated with the first wearable device 104-a may be used as inputs to the second wearable device 104-b, and/or may be used to analyze or otherwise interpret data collected by the second wearable device 104-b. Conversely, the data/capabilities associated with the second user device 104-b may be used as inputs to the first wireless device 104-a, and/or may be used to analyze or otherwise interpret data collected by the first wireless device 104-a.

As noted previously herein, the first wearable device 104-a may be configured to collect physiological data and/or user inputs (e.g., gestures) from the user, where the second wearable device 104-b may be configured to acquire environmental data associated with an environment of the user. The environmental data may include, but is not limited to, images of the user's environment (captured via the image capture device 320), audio data of the user's environment (e.g., ambient noise of the user's environment, voice commands from the user's environment, etc.). In some aspects, gestures/user inputs received from one device may be used to trigger actions on the other device. Further, the wearable system 300 may be configured to compare or correlate the physiological data collected via the first wearable device 104-a with the environmental data collected via the second wearable device 104-b to determine specific impacts or effects that the user's environment has on the user's physiological data and overall health.

Referring to the first wearable device 104-a in FIG. 3, the one or more biometric sensors 305 used for health monitoring/authentication capabilities, one or more motion sensors 310 used for gesture/user input capabilities, and one or more input components 315 used for user input capabilities. For example, the input component may include mechanical or capacitive buttons. By way of another example, in some cases, the outer cover (e.g., outer housing 205-b) of the wearable ring device may be configured to rotate relative to the rest of the housing, where rotation of the outer housing 205-b (or a portion of the outer housing) may be used as an input.

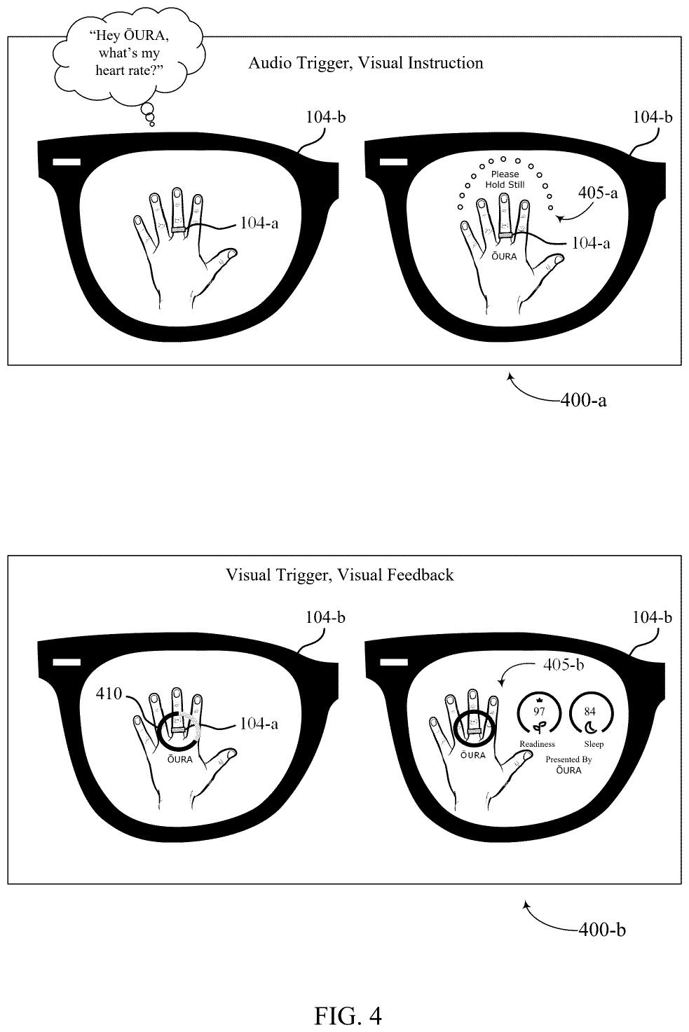

Comparatively, the second wearable device 104-b may include one or more image capture devices 320 (e.g., cameras, video cameras, etc.) for sight/imaging capabilities, one or more audio capture devices 325 (e.g., microphones) for hearing/user input capabilities (e.g., to receive voice commands from the user), one or more speakers 330 for voice/user feedback capabilities (e.g., to provide audio feedback to the user), and a display 335 for visual feedback (e.g., lenses of smart glasses can display augmented reality (AR), virtual reality (VR), and/or mixed reality images/information). For example, as will be described in further detail with respect to FIG. 4, the second wearable device 104-b (e.g., smart glasses) may be configured to generate and display (via display 335) information that is overlaid on top of a view that the user sees/observes through the lenses of the smart glasses (such as an AR visualization of the wearable application 250 associated with the first wearable device 104-a).

In this regard, the lenses of the smart glasses (through which the user sees through the smart glasses) may include or otherwise be integrated with a display device. In such cases, when there is no data to be displayed or overlaid via the display 335, the lenses of the smart glasses may act as normal lenses. However, when there is data to be displayed, the smart glasses may activate the display 335 such that the data may be overlaid or otherwise displayed on a surface of the lenses (e.g., AR visualization that is “overlaid” on top of the user's real-world view through the lenses). In some aspects, the AR visualization may be projected onto the lenses of the smart glasses (e.g., display 335 includes a projection device that projects an AR visualization onto the lenses of the smart glasses). In other cases, the lenses may include embedded displays (e.g., display 335) that are embedded within, or otherwise integrated into, the lenses themselves.

The one or more biometric sensors 305 may be configured to acquire physiological data from the user, such as PPG sensors (e.g., LEDs, PDs), temperature sensors, electrocardiogram (ECG) sensors, pressure sensors, and the like. As such, the one or more biometric sensors may be used for health monitoring (e.g., sleep tracking) and/or for authentication purposes (e.g., to authenticate an identity of the user for payments, access, etc.). The one or more motion sensors 310 (e.g., gyroscopes, accelerometers) may be used to acquire motion data from the user. Motion data collected via the wearable ring device 104-a may be used to evaluate user activities (e.g., activity detection/classification, sleep monitoring), and to identify gestures performed by the user, where identified gestures may be used as user inputs/commands.

Similarly, in some cases, the first wearable device 104-a may include one or more dedicated input components 315 that are configured to receive user inputs/commands from the user. The input components 315 may include, but are not limited to, a physical button, a capacitive button, a capacitive touch pad, conductive electrodes, force/deformation sensors, rotational components, accelerometer/impact-based gestures, fingerprint sensors, RF communication components, photovoltaic cells/light sensors, microphones for audio inputs, and the like. The various types of input components 315 may be configured to receive user inputs/commands in various manners.

In some aspects, gestures/inputs received via the first wearable device 104-a may be used to trigger certain actions at (or by) the user device 106 and/or the second wearable device 104-b. For example, in some cases, a gesture/user input identified by the first wearable device 104-a may cause the wearable smart glasses to take an image. In this regard, the gesture recognition capabilities of the first wearable device 104-a may be used to facilitate operation of the second wearable device 104-b.

For example, a user may press or contact physical/capacitive buttons to initiate a user input/command, including multi-tap patterns where the quantity and timing of the multiple taps is used as part of the user input (e.g., program a specific tap sequence to trigger an action). A capacitive touch pad may be used to identify directional inputs using swipes and multi-touch patterns. Conductive electrodes (e.g., electrodes used for ECG/BioZ measurements) may also be used to detect when the circuit is closed when the user touches the electrode(s), similar to a capacitive button. Further, conductive electrodes could possibly be used to indicate direction (to differentiate between user inputs/commands) by swiping across two or three electrodes on the surface of the first wearable device 104-a, where the first wearable device 104-a is configured to identify the direction and the timing of the circuits being open/closed as the user's finger slides over the multiple contacts.

In some cases, the input component 315 may include force or deformation sensors, where the user taps or squeezes the first wearable device 104-a to exert a force used to deform a portion of the first wearable device 104-a. In other cases, the first wearable device 104-a may be able to identify rotation of the ring as a user input. For example, the user may be able to rotate the first wearable device 104-a while it is being worn on the user's finger, where input components 315, motion sensors 310 (e.g., accelerometers, IMU sensors), and/or optical sensors of the first wearable device 104-a may be configured to identify the rotation(s) and recognize the rotation(s) as a user input. For instance, the user may be able to define different rotational patterns to indicate different actions similar to combination-dial safes (e.g., Action #1=2 rotations clockwise+2 rotations counterclockwise+1 rotation clockwise).

In some cases, the input component 315 may include accelerometer/impact-based components configured to recognize gestures from the user, such as rapping knuckles on a table, clapping, clicking fingers, first bump, bumping two devices together, etc.). In other cases, the input component 315 may enable the first wearable device 104-a to be used as an “air mouse,” where the first wearable device 104-a identifies movements of the user's hand. In such cases, identified movement of the user's hand may be directly translated into movement of object or cursor, like a game motion controller or direct control of a drone (e.g., raise hand up/down to change altitude, twist left/right to yaw, tilt up/down to pitch, move hand in 3D space to change position, etc.). Similarly, movements/gestures of the user's hand (which are identified via the first wearable device 104-a) may be used to move a cursor or otherwise interact with graphical elements shown on the display 335 of the second wearable device 104-b. For instance, in some aspects, the wearable device 104-a may be configured to recognize movements of the user's hand as “air gestures,” or pre-trained gestures such as swipes, pushes, waves, etc. performed by the hand/arm in the air. In some cases, the first wearable device 104-a may be configured to recognize unique air gestures that may be pre-trained by the user, such as an “air signature” that may be used for identity/authentication purposes.

In additional or alternative implementations, components of the system 300 (e.g., wearable devices 104-a, 104-b, user device 106, servers, etc.) may be configured to identify or otherwise recognize gestures performed by the user based on PPG data collected via the wearable device 104-a. Such PPG data may be used in addition to, or in the alternate to, acceleration/movement data in order to identify gestures performed by the user. In particular, gestures performed by the user may cause or induce changes in PPG data collected via the first wearable device 104-a. That is, movement of the user's hand (e.g., waving the hand back and forth) may cause changes in the blood flow through the vessels in the hand, which may thereby be detected via acquired PPG data. In this regard, the system may be configured to recognize certain patterns or changes in the PPG data, and may identify or otherwise recognize gestures performed by the user based on the identified patterns/changes in the PPG data.

In some implementations, gestures and/or movements of the user's hand may be identified using both information collected by the first wearable device 104-a (e.g., acceleration data, etc.), as well as information collected by the second wearable device 104-b (e.g., images/video of the user's hand captured via the second wearable device 104-b). In such cases, the system may be configured to correlate motion data (e.g., acceleration data) collected via the first wearable device 104-a with image/video data collected via the second wearable device 104-b in order to identify gestures performed by the user. Using data from multiple sources may enable more accurate and reliable gesture recognition.

In some cases, gestures identified via the first wearable device 104-a (and/or second wearable device 104-b) may be combined with sensors in other objects, or with an AR space representation (e.g., as used in AR headsets). For example, the user may be able to point to an object/device (e.g., TV, drone, etc.) to control the object/device. For instance, the user may be able to point at their TV (e.g., using their hand/finger used to wear the smart ring), where the first wearable device 104-a and/or the second wearable device 104-b may be configured to identify that the user is pointing at the TV (e.g., via motion data and/or RF signals at the first wearable device 104-a, via image data of the user's hand/ring collected via the smart glasses). Upon identifying that the user is pointing at the TV, the first wearable device 104-a, the second wearable device 104-b, the user device 106, or any combination thereof, may transmit signals to establish a wireless connection with the TV such that subsequent gestures (as identified via either wearable device 104) may be used to control the TV. Similarly, as noted above, identified gestures may be used to control a cursor or otherwise select data objects displayed via the display 335 of the second wearable device 104-b (e.g., adjust/control an AR visualization that is overlaid on the lenses of the smart glasses).

In some aspects, the input component(s) 315 may be configured to identify gestures performed by deforming the hand or fingers, or touching palm/fingers together (e.g., pinch 2/3/4 fingers, open/clench fist, etc.). In such cases, the user may be able to pre-define such gestures to indicate certain actions. Such hand deformation gestures may be identified using optical or electrical sensing of muscle and blood activity, with or without using motion data collected via the motion sensors 310. Further, gestures may be identified via image/video data collected via the image capture devices 320 of the second wearable device 104-b.

In some aspects, the input component(s) 315 may include fingerprint sensors used to detect finger touch as a user input. The fingerprint sensors may be used as part of normal fingerprint sensor functionality to activate the sensor, but can be used to identify touch inputs. In other cases, photovoltaic cells or light sensors may be used to detect finger touch as user inputs/commands. For example, the first wearable device 104-a may identify a pattern of on/off ambient light obstruction due to the user tapping a light sensor in a specific, pre-defined tap pattern that corresponds to (e.g., indicates) a specific gesture/action.

In some aspects, the input component(s) 315 may include RF communication components that are configured to recognize gestures upon receiving RF signals. For example, the user may move the first wearable device 104-a close to (e.g., touching) another device (e.g., second wearable device 104-b) with an RF component, where signals received from the RF component of the other device are recognized as a gesture. For instance, a user may wear a wearable ring device on each hand, and may touch the rings together (e.g., clasp hands, bump fists, clap) to indicate a gesture/command. Similar gestures/commands may be recognized by the user bringing the ring into close proximity with another on-body device, such as a patch or smart glasses (e.g., second wearable device 104-b) that activates when the user taps/pats the patch/smart glasses with the smart ring. Further, similar gestures/commands may be recognized by two different users bringing their rings together (e.g., two different users fist-bumping one another to share contact information or perform some other action). In some cases, RF-based gestures described herein may be used in conjunction with other motion and/or physiological triggers as inputs (e.g., gesture recognized when RF signals are received in conjunction with a motion pulse caused by touching rings together).

Further, in some cases, physiological data collected via the biometric sensors 305 may be used as an input or trigger for the second wearable device 104-b. In particular, the system (e.g., second wearable device 104-b) may identify a satisfaction of one or more trigger conditions for collecting image/audio data based on the physiological data collected by the first wearable device 104-a. For instance, the second wearable device 104-b may be configured to take an image or perform some other action when the user's heart rate or respiration rate exceeds some threshold. Any physiological measurement or calculation may be used as an input. For example, the first wearable device 104-a (and/or user device 106) may be configured to calculate a Stress Score associated with the user based on acquired physiological data, where the Stress Score may be used to trigger actions/inputs via the second wearable device 104-b. In some aspects, the system 300 (e.g., first wearable device 104-a, second wearable device 104-b, user device 106, servers, etc.) may compare physiological data collected via the first wearable device 104-a (and/or the second wearable device 104-b) to multiple thresholds and/or trigger conditions, where satisfaction of different trigger conditions causes the second wearable device 104-b to perform different actions or functions (e.g., satisfaction of a first trigger condition causes the smart glasses to capture and/or save/store additional image data, where satisfaction of a second trigger condition causes the smart glasses to capture and/or save/store additional audio data).

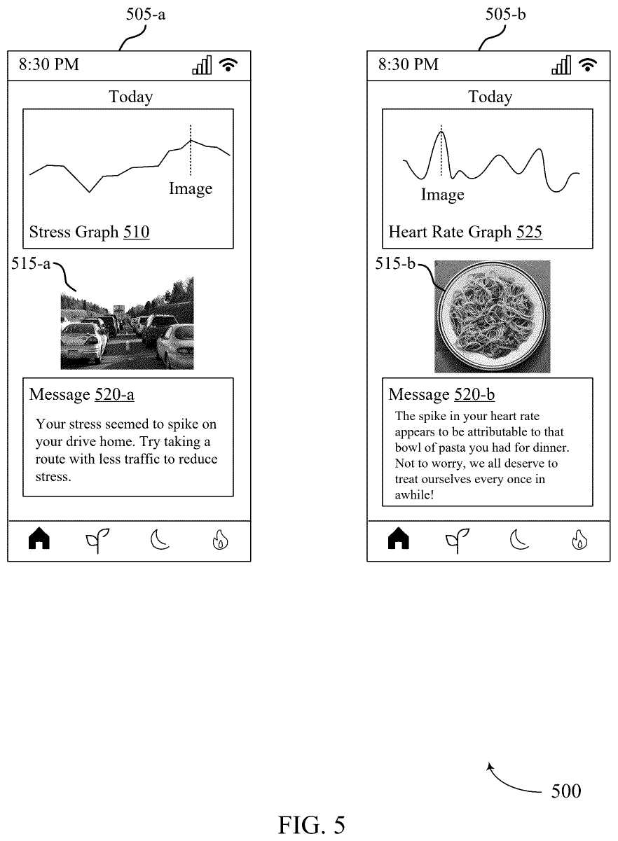

For instance, the second wearable device 104-b may be configured to record audio or take an image when the user's Stress Score exceeds some value. By triggering the second wearable device 104-b to take images and/or collect audio when the user's Stress Score exceeds some threshold, the wearable system 300 may be able to analyze the collected images/audio to better evaluate the root causes of the user's rising stress. For instance, by triggering the second wearable device 104-b to take images when the user's Stress Score spikes, the system may be able to identify or “learn” that traffic or certain foods (as determined by images taken via the second wearable device 104-b) may be the cause of the user's stress. In such cases, image/audio data collected via the second wearable device 104-b may be uploaded to the wearable application 250, time-stamped, and correlated with corresponding physiological data collected via the first wearable device 104-a (as will be further shown and described with reference to FIG. 5). It is noted herein that the second wearable device 104-b may be configured to continuously capture audio and/or image data (e.g., to identify gestures or verbal commands performed by the user). However, in some cases, the second wearable device 104-b may not be configured to store or otherwise save all the collected audio/image data (e.g., to reduce memory requirements, processing requirements, etc.). For instance, the second wearable device 104-b may only store the most recent 30 seconds of collected data at a time (and may continually discard or delete data that is older than 30 seconds). Accordingly, in such cases, gestures or other commands (e.g., verbal commands) detected by the system may trigger the second wearable device 104-b to save or otherwise store the audio/image data collected by the second wearable device 104-b (e.g., save/store data that would otherwise be discarded or otherwise not saved). For instance, upon detecting a gesture or voice command, the second wearable device 104-b may save/store the audio/image data collected some X time prior to the detected gesture/command, and/or some Y time subsequent to the detected gesture/command (where X and Y may be preconfigured by the system, defined by the user, etc.). In this regard, gestures and commands may be used to store/save data collected by the second wearable device 104-b during a time interval over which the gesture/command was performed, which enables the stored audio/image data to be correlated with the physiological data collected by the wearable ring device 104-a (e.g., to identify how events, conditions, and/or circumstances observed in the saved audio/image data affected the user's physiological data).

As noted previously herein, the first wearable device 104-a may be configured to identify certain gestures/user inputs, where such gestures/user inputs may be used as inputs to trigger certain actions on/by the first wearable device 104-a, the user device 106, the second wearable device 104-b, or any combination thereof. Table 1 below illustrates multiple use-cases where gestures/inputs received via the first wearable device 104-a (e.g., wearable ring device) may be used to trigger actions by the second wearable device 104-b (e.g., wearable smart glasses).

TABLE 1
Use cases for using data from smart ring to trigger actions by smart glasses

| | | Output (performed by |
|---|---|---|
| | Input (received via first | second wearable device |
| Use Case | wearable device 104-a) | 104-b) |
| Activity Companion | Activity tracking | Real-time audio |
| Start and stop a workout | (collected physiological | confirmation; |
| with smart ring; smart | data); | Audio and/or visual |
| glasses provide audio | AAD (Automatic Activity | content |
| confirmation; optionally, | Detection) | |
| play guided content for | | |
| guided workout | | |
| Context-Aware Tagging | On-ring interaction (e.g., | Photo capture |
| Trigger photo capture | tap ring) | |
| using the smart ring to | | |
| indicate that a photo taken | | |
| by the smart glasses | | |
| should be added as a tag in | | |
| the wearable application | | |
| (e.g., habit tracking, | | |
| journaling) | | |
| Meal Tracking | On-ring interaction (e.g., | Photo capture |
| Trigger photo capture | tap ring) | |
| using the smart ring to | | |
| indicate that a photo taken | | |
| by the smart glasses | | |
| should be added as a meal | | |
| in the wearable application | | |
| Programmable Health | Biometric sensing | Audio and/or visual |
| Alerts | (collected physiological | notifications; |
| User is notified when there | data) | Audio and/or visual |
| are signs of stress (e.g., | | content |
| sudden HR increase) with | | |
| the option to initiate an | | |
| meditation | | |
| Contactless Payments | Payment features (e.g., | Audio/visual |
| User initiates contactless | signals and/or gestures | confirmation; |
| payment over POS with | identified via smart ring | Additional data collection |
| smart ring, smart glasses | and/or smart glasses to | used to complete user |
| provide added | trigger | authentication |
| authorization or audio | authentication/payment) | |
| feedback | | |
| User-Assigned Actions | On-ring interaction (e.g., | Actions compatible with |
| Discreet and customizable | tap) | smart glasses and partner |
| actions for compatible | | apps (e.g., music control, |
| apps: music controls, take | | scroll, swipe, etc.) |
| photos, live stream | | |
| controls, presentation | | |
| controls etc. | | |

In addition to gestures at the first wearable device 104-a being used to trigger actions at the second wearable device 104-b, the inverse may also be used where data collected via the second wearable device 104-b may be used to adjust or trigger actions at the first wearable device 104-a. That is, image/audio data associated with the environment of the user collected via the second wearable device 104-b may be used to adjust measurement parameters at the first wearable device 104-a, such as a measurement periodicity, LED intensity, or both. For instance, image data collected via the second wearable device 104-b may be used to determine that the user is outdoors with high ambient light levels, where such location/ambient light data may then be used to increase an LED intensity at the first wearable device 104-a (to enable high quality data collection in high ambient light conditions). By way of another example, image/audio data collected via the second wearable device 104-b may be used to determine that the user is sitting in traffic, or is in a crowded room. In this example, such environmental information (e.g., location of the user in traffic or in a crowded room) may be used to trigger stress-related measurements at the first wearable device 104-a.

In some implementations, data collected via the first wearable device 104-a, the second wearable device 104-b, or both, may be used to authenticate an identity of the user, where user authentication may be used to trigger certain actions (e.g., open a door, authorize a payment/transaction, etc.). For example, in some cases, PPG data collected via the biometric sensors 305 of the first wearable device 104-a may be used in conjunction with additional data collected via the second wearable device 104-b (e.g., voice data, retinal scans captured via the image capture devices 320, images of a pre-defined object, etc.) in order to provide a sort of “two-factor authentication” for the user. In some cases, the user may be able to define custom authentication conditions that will be used to authenticate the user (e.g., authenticate the user's identity when the user's PPG/ECG data matches corresponding PPG/ECG profiles, and/or when the image capture device 320 captures an image of this hand sign/signal). Upon authenticating the identity of the user, the first wearable device 104-a, the second wearable device 104-b, the user device 106, or any combination thereof, may transmit a signal to an external device (e.g., door to access a building, door to a vehicle, point-of-service device, financial kiosk, vending machine, etc.), where the signal includes an authentication of the user (e.g., confirmation of the user's identity) and causes the external device to perform some action (e.g., open the door, authorize a payment/transaction, dispense funds or goods, etc.).

There may be several different procedures, implementations, or flows for performing authentication in accordance with aspects of the present disclosure. For example, in the context of authentication for payments, the authentication of the user may be performed entirely on the wearable device 104 and/or user device 106 associated with the user (without any signal/request from an external device, such as a payment terminal). In this example, the transaction functionality may be “unlocked” on/at the wearable device 104/user device 106 based on the authentication of the user. That is, the capability of the wearable device 104/user device 106 to transmit a signal to verify/authorize the transaction may be “unlocked” based on authenticating the identity of the user. Conversely, if authentication of the user is not performed or successfully completed, subsequent transaction attempts will fail at the external reader (e.g., because the wearable device 104 and/or user device 106 did not provide the signaling/information to authorize the transaction).

In accordance with a first “flow” for authentication, the system 300 (e.g., first wearable device 104-a, second wearable device 104-b, user device 106, servers, etc.), may combine environmental data collected via the second wearable device 104-b (e.g., smart glasses) with data collected from the first wearable device 104-a (e.g., ring) to authenticate the user and unlock further functionality on one (or both) of the wearable devices (e.g., unlock functionality of the glasses for interacting with external devices, unlock functionality for performing/authorizing transactions or payments digitally or online or via physical RF radio signals transmitted to a payments terminal, etc.).

Comparatively, in accordance with a second “flow” for authentication, the second wearable device 104-b (e.g., smart glasses) may communicate captured environmental data to the first wearable device 104-a, where the first wearable device 104-a may combine the received environmental data with its own collected data (e.g., PPG data, temperature data, etc.) to authenticate the user and “unlock” further functionality on the ring, as described herein.

FIG. 4 show example wearable use cases 400-a, 400-b that supports techniques for multiple wearable devices in accordance with aspects of the present disclosure. Aspects of the wearable use cases 400 may implement, or be implemented by, aspects of the system 100, the system 200, the wearable system 300, or any combination thereof.

As noted previously herein, the first wearable device 104-a (e.g., smart ring) and the second wearable device 104-b (e.g., smart glasses) may be used in conjunction with one another to facilitate various use cases and capabilities within the wearable system 300. That is, data collected by the various wearable devices 104-a, 104-b may be exchanged between one another to enable various capabilities and use cases that would otherwise not be possible with the individual wearable devices 104 on their own.

For example, the first wearable use case 400-a illustrates an example where a user uses a voice prompt to ask the second wearable device 104-b (e.g., smart glasses, or application/program executable via the smart glasses, such as ChatGPT, MetaAI, etc.) to check the user's heart rate. The user states: “Hey Oura, what's my heart rate?,” where the user's voice is recognized via the audio capture device 325 of the second wearable device 104-b. In this example, the user may position their hand with the first wearable device 104-a in the frame of view of the second wearable device 104-b, as shown. The second wearable device 104-b may identify the first wearable device 104-a within the frame of view, and present an AR visualization 405-a that instructs the user to keep their hand still to enable the first wearable device 104-a to collect an accurate heart rate measurement. As such, the second wearable device 104-b may display the AR visualization 405-a guiding them through the process so that the user knows how long to hold their hand still for.

The AR visualization 405-a may be displayed or otherwise overlaid onto or within the lenses of the second wearable device 104-b (e.g., projected onto the side of the lenses facing the user's eyes, or displayed via a display(s) integrated/embedded into the lenses themselves). In this regard, the AR visualization 405-a may be “overlaid” on top of the view of the user's surroundings that the user sees through the lenses of the second wearable device 104-b. In some aspects, the second wearable device 104-b may acquire or otherwise receive the data used to generate the AR visualization 405-a directly from the first wearable device 104-a, from the user device 106, or both. For example, in some cases, the second wearable device 104-b may access the health-related application (e.g., wearable application 250) by establishing a wireless connection with the user device 106, where the AR visualization 405-a may include an instance or version of the health-related application that is accessible via the connection with the user device 106. For instance, the second wearable device 104-b may access the wearable application 250 via a wireless connection with the user device 106 upon identifying that the first wearable device 104-a in the frame of view of the second wearable device 104-b, as shown.

In additional or alternative implementations, the second wearable device 104-b may be configured to display information associated with the first wearable device 104-a without any audio/voice commands. For example, referring to the second wearable use case 400-a, the wearable system may support an example that uses both wearable devices 104-a, 104-b for image and gesture recognition. In this example, the user may position their hand wearing the first wearable device 104-a in the frame of view (line of sight) of the second wearable device 104-b. The second wearable device 104-b may automatically recognize the first wearable device 104-a in the frame of view, and may show an AR progress bar 410 to signal how long the user should hold their hand still to bring up an AR version (e.g., AR experience) of the wearable application 250. Once the AR experience of the wearable application 250 has been loaded/launched (as indicated via completion of the AR progress bar 410), the second wearable device 104-b may display an AR visualization 405-b that shows a visual depiction of various metrics/information associated with the wearable application 250 (e.g., Sleep Score, Readiness Score, Activity Score, Stress Score, heart rate, SpO2, etc.). In some cases, the user may perform gestures that are recognized by the first wearable device 104-a and/or the second wearable device 104-b to swipe/scroll through various metrics or information within the AR visualization 405-b (e.g., AR experience) of the wearable application 250. In some cases, the user may customize what data or pages are included within the AR visualization 405-b viewed via the second wearable device 104-b.

In some aspects, the AR visualization 405-b may display “live” measurements (e.g., real-time or near-real time measurements) that are currently being performed (or have recently been performed) by the first wearable device 104-a, such as live heart rate measurements, the heart rate measurements, and the like. In this regard, the user may be able to quickly glance at their ring (through their smart glasses) to view their heart rate in real-time or near-real time. In some aspects, the second wearable device 104-b may instruct the first wearable device 104-a to trigger “live” measurements based on identifying that the first wearable device 104-a is depicted or captured within the image data collected by the second wearable device 104-b. That is, upon determining that the smart ring is depicted within the images captured via the image capture devices 320, the smart glasses may transmit a signal to the smart ring to cause the smart ring to begin performing measurements (e.g., “live” heart rate and/or SpO2 measurements), where the smart ring can then relay the “live” measurements back to the smart glasses (for display via the AR visualization 405-b) in real-time or near-real time.

In addition to simply identifying that the first wearable device 104-a is depicted within images captured by the second wearable device 104-b, the second wearable device 104-b may be configured to analyze collected image data (e.g., video data) to identify gestures performed by the user, such as hand gestures. In some cases, gestures may be identified using motion data collected via the first wearable device 104-a in conjunction with image data collected via the second wearable device 104-b. In some cases, the second wearable device 104-b may be configured to display the AR visualization 405-b (and/or change information displayed via the AR visualization 405-b) based on identified gestures.

FIG. 5 shows an example of a GUI 500 that supports techniques for multiple wearable devices in accordance with aspects of the present disclosure. Aspects of the GUI 500 may implement, or be implemented by, aspects of the system 100, the system 200, the wearable system 300, the wearable use cases 400, or any combination thereof.

The GUI 500 in FIG. 5 illustrates application pages 505-a, 505-b that may be displayed on a screen or display (e.g., display 335) of the user device 106 and/or the second wearable device 104-b via an application (e.g., wearable application 250), as described herein, based on instructions received from one or more processors (e.g., of a server 110, the user device 106, a wearable device 104, or another device). For example, the application pages 505 may illustrate applications of a health-related application that is associated with the first wearable device 104-a (e.g., wearable ring device) and executable by the user device 106. In such cases, the health-related application depicted via the application pages 505 may be accessible by the second wearable device 104-b (e.g., smart glasses), where the AR visualizations 405 shown and described in FIG. 4 may include instances (or versions) of the health-related application depicted via the application pages 505.

As shown in FIG. 5, the application pages 505 of the wearable application 250 may display information/data collected via the first wearable device 104-a, the second wearable device 104-b, the user device 106, an external device (e.g., charger device, smart appliance, virtual assistant), or any combination thereof. For example, the first application page 505-a may display a stress graph 510 that shows how the user's Stress Score changes throughout the day, where the Stress Scores of the stress graph 510 are calculated based on physiological data collected via the first wearable device 104-a (e.g., heart rate data, temperature data, respiration rate data, SpO2 data, etc.).

In some aspects, environmental data collected via the second wearable device 104-b (e.g., image/audio data associated with the surrounding environment of the user) may be used by the system to further evaluate/analyze the physiological data collected via the first wearable device 104-a. In particular, the system may evaluate environmental data collected via the second wearable device 104-b (e.g., image/audio data) to identify physiological effects/impacts that the user's surrounding environment has on their physiological data and overall health.

For example, the first application page 505-a may display an image 515-a captured via the second wearable device 104-b, as well as a point in time within the stress graph 510 that indicates when the image 515-a was captured. As noted previously herein, the second wearable device 104-b may be configured to capture the image 515-a (and/or load the image to the wearable application 250) based on a gesture from the user, based on an automated trigger (e.g., the user's Stress Score or other physiological measurement satisfying some threshold), or both. In this example, the wearable system 300 (e.g., servers 110, user device 106, etc.) may identify that the image 515-a depicts an image of the user sitting in traffic, which may be a stressful situation for many users. As such, the wearable system 300 may “learn” that traffic may cause a spike in the user's Stress Score (as illustrated via the stress graph 510), and may display a message 520-a to the user stating “Your stress seemed to spike on your drive home. Try taking a route with less traffic to reduce stress.”

By way of another example, the second application page 505-b may display a heart rate graph 525 illustrating how the user's heart rate changes throughout the day. The application page 505-b may additionally show an image 515-b captured via the second wearable device 104-b, as well as a point in time within the heart rate graph 525 that indicates when the image 515-b was captured. As noted previously herein, the second wearable device 104-b may be configured to capture the image 515-b (and/or load the image to the wearable application 250) based on a gesture from the user, based on an automated trigger (e.g., the user's heart rate or other physiological measurement satisfying some threshold), or both. In this example, the wearable system 300 (e.g., servers 110, user device 106, etc.) may identify that the image 515-b depicts a plate of pasta. As such, the wearable system 300 may “learn” how different foods may affect the user's physiological data, including the user's heart rate, and may display a message 520-b to the user stating “The spike in your heart rate appears to be attributable to that bowl of pasta you had for dinner. Not to worry, we all deserve to treat ourselves every once in awhile!”

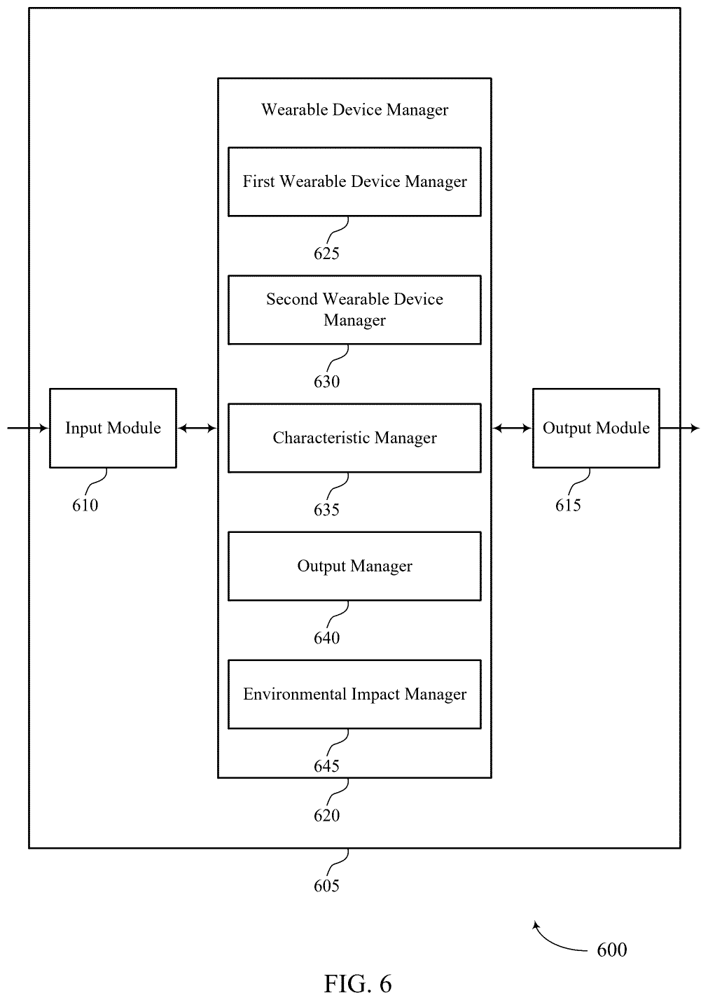

FIG. 6 shows a block diagram 600 of a device 605 that supports techniques for multiple wearable devices in accordance with aspects of the present disclosure. The device 605 may include an input module 610, an output module 615, and a wearable device manager 620. The device 605, or one or more components of the device 605 (e.g., the input module 610, the output module 615, the wearable device manager 620), may include at least one processor, which may be coupled with at least one memory, to support the described techniques. Each of these components may be in communication with one another (e.g., via one or more buses).

For example, the wearable device manager 620 may include a first wearable device manager 625, a second wearable device manager 630, a characteristic manager 635, an output manager 640, an environmental impact manager 645, or any combination thereof. In some examples, the wearable device manager 620, or various components thereof, may be configured to perform various operations (e.g., receiving, monitoring, transmitting) using or otherwise in cooperation with the input module 610, the output module 615, or both. For example, the wearable device manager 620 may receive information from the input module 610, send information to the output module 615, or be integrated in combination with the input module 610, the output module 615, or both to receive information, transmit information, or perform various other operations as described herein.

The first wearable device manager 625 may be configured as or otherwise support a means for acquiring physiological data from a user via a first wearable device worn at a first position on a body of the user, the physiological data comprising heart rate data, respiration rate data, motion data, or any combination thereof. The second wearable device manager 630 may be configured as or otherwise support a means for acquiring environmental data associated with one or more characteristics of an environment of the user via a second wearable device worn at a second position on the body of the user, the environmental data comprising image data, audio data, or both. The characteristic manager 635 may be configured as or otherwise support a means for identifying one or more physiological characteristics of the user, one or more characteristics of the environment of the user, or both, based at least in part on the physiological data and the environmental data, respectively. The output manager 640 may be configured as or otherwise support a means for causing the first wearable device to adjust one or more measurement characteristics used to acquire the physiological data based at least in part on the one or more characteristics of the environment of the user, or cause the second wearable device to acquire additional environmental data based at least in part on the one or more physiological characteristics of the user.

The first wearable device manager 625 may be configured as or otherwise support a means for acquiring physiological data from a user via a first wearable device worn at a first position on a body of the user, the physiological data comprising heart rate data, respiration rate data, motion data, or any combination thereof. The second wearable device manager 630 may be configured as or otherwise support a means for acquiring environmental data associated with one or more characteristics of an environment of the user via a second wearable device worn at a second position on the body of the user, the environmental data comprising image data, audio data, or both. The environmental impact manager 645 may be configured as or otherwise support a means for identifying one or more impacts that the environment of the user had on the physiological data of the user based at least in part on correlating a first portion of the physiological data collected during a time interval with a first portion of the image data, the audio data, or both, collected during the time interval. The output manager 640 may be configured as or otherwise support a means for generating one or more signals configured to cause a user device, the second wearable device, or both, to provide a message comprising data associated with the one or more impacts.

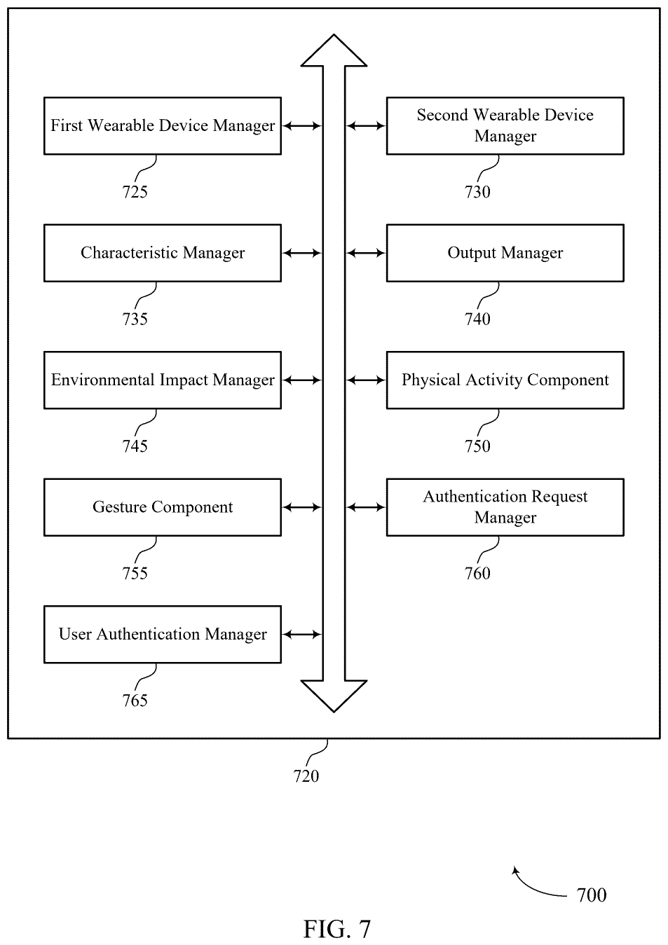

FIG. 7 shows a block diagram 700 of a wearable device manager 720 that supports techniques for multiple wearable devices in accordance with aspects of the present disclosure. The wearable device manager 720 may be an example of aspects of a wearable device manager or a wearable device manager 620, or both, as described herein. The wearable device manager 720, or various components thereof, may be an example of means for performing various aspects of techniques for multiple wearable devices as described herein. For example, the wearable device manager 720 may include a first wearable device manager 725, a second wearable device manager 730, a characteristic manager 735, an output manager 740, an environmental impact manager 745, a physical activity component 750, a gesture component 755, an authentication request manager 760, a user authentication manager 765, or any combination thereof. Each of these components, or components of subcomponents thereof (e.g., one or more processors, one or more memories), may communicate, directly or indirectly, with one another (e.g., via one or more buses).

The first wearable device manager 725 may be configured as or otherwise support a means for acquiring physiological data from a user via a first wearable device worn at a first position on a body of the user, the physiological data comprising heart rate data, respiration rate data, motion data, or any combination thereof. The second wearable device manager 730 may be configured as or otherwise support a means for acquiring environmental data associated with one or more characteristics of an environment of the user via a second wearable device worn at a second position on the body of the user, the environmental data comprising image data, audio data, or both. The characteristic manager 735 may be configured as or otherwise support a means for identifying one or more physiological characteristics of the user, one or more characteristics of the environment of the user, or both, based at least in part on the physiological data and the environmental data, respectively. The output manager 740 may be configured as or otherwise support a means for causing the first wearable device to adjust one or more measurement characteristics used to acquire the physiological data based at least in part on the one or more characteristics of the environment of the user, or cause the second wearable device to acquire additional environmental data based at least in part on the one or more physiological characteristics of the user.

In some examples, the physical activity component 750 may be configured as or otherwise support a means for identifying that the user is engaged in a physical activity based at least in part on the physiological data collected via the first wearable device. In some examples, the physical activity component 750 may be configured as or otherwise support a means for determining an activity type of the physical activity based at least in part on the physiological data collected via the first wearable device and the environmental data acquired via the second wearable device.

In some examples, the gesture component 755 may be configured as or otherwise support a means for identifying a gesture performed by the user based at least in part on the motion data collected via the first wearable device. In some examples, the first wearable device manager 725 may be configured as or otherwise support a means for causing the second wearable device to save a portion of the image data, save a portion of the audio data, collect additional image data, collect additional audio data, or any combination thereof, based at least in part on identifying the gesture.

In some examples, the authentication request manager 760 may be configured as or otherwise support a means for receiving, from an external device, a request to authenticate the user. In some examples, the user authentication manager 765 may be configured as or otherwise support a means for authenticating an identity of the user based at least in part on the physiological data collected via the first wearable device and the environmental data collected via the second wearable device. In some examples, the output manager 740 may be configured as or otherwise support a means for outputting a message to the external device comprising an authentication of the user based at least in part on authenticating the identity of the user.

In some examples, the one or more characteristics of the environment of the user comprise a location of the user, an ambient light level, an ambient noise level, a voice of the user, or any combination thereof.

In some examples, the first wearable device comprises a wearable ring device, a wrist-worn wearable device, or both. In some examples, the second wearable device comprises wearable smart glasses.

In some examples, the first wearable device manager 725 may be configured as or otherwise support a means for acquiring physiological data from a user via a first wearable device worn at a first position on a body of the user, the physiological data comprising heart rate data, respiration rate data, motion data, or any combination thereof. In some examples, the second wearable device manager 730 may be configured as or otherwise support a means for acquiring environmental data associated with one or more characteristics of an environment of the user via a second wearable device worn at a second position on the body of the user, the environmental data comprising image data, audio data, or both. The environmental impact manager 745 may be configured as or otherwise support a means for identifying one or more impacts that the environment of the user had on the physiological data of the user based at least in part on correlating a first portion of the physiological data collected during a time interval with a first portion of the image data, the audio data, or both, collected during the time interval. In some examples, the output manager 740 may be configured as or otherwise support a means for generating one or more signals configured to cause a user device, the second wearable device, or both, to provide a message comprising data associated with the one or more impacts.

In some examples, the characteristic manager 735 may be configured as or otherwise support a means for identifying one or more characteristics of the environment of the user based at least in part on the image data, the audio data, or both, collected via the second wearable device. In some examples, the first wearable device manager 725 may be configured as or otherwise support a means for causing the first wearable device to adjust one or more measurement characteristics used to acquire the physiological data based at least in part on the one or more characteristics of the environment of the user, wherein the first wearable device is configured to acquire additional physiological data based at least in part on adjusting the one or more measurement characteristics.

In some examples, the characteristic manager 735 may be configured as or otherwise support a means for identifying one or more physiological characteristics of the user based at least in part on the image data, the audio data, or both, collected via the second wearable device. In some examples, the second wearable device manager 730 may be configured as or otherwise support a means for causing the second wearable device to acquire additional image data, additional audio data, or both, based at least in part on the one or more physiological characteristics of the user.

In some examples, the physical activity component 750 may be configured as or otherwise support a means for identifying that the user is engaged in a physical activity based at least in part on the physiological data collected via the first wearable device. In some examples, the physical activity component 750 may be configured as or otherwise support a means for determining an activity type of the physical activity based at least in part on the physiological data collected via the first wearable device and the environmental data acquired via the second wearable device.

In some examples, the gesture component 755 may be configured as or otherwise support a means for identifying a gesture performed by the user based at least in part on the motion data collected via the first wearable device. In some examples, the output manager 740 may be configured as or otherwise support a means for causing the second wearable device to save a portion of the image data, save a portion of the audio data, collect additional image data, collect additional audio data, or any combination thereof, based at least in part on identifying the gesture.

In some examples, the authentication request manager 760 may be configured as or otherwise support a means for receiving, from an external device, a request to authenticate the user. In some examples, the user authentication manager 765 may be configured as or otherwise support a means for authenticating an identity of the user based at least in part on the physiological data collected via the first wearable device and the environmental data collected via the second wearable device. In some examples, the output manager 740 may be configured as or otherwise support a means for outputting a message to the external device comprising an authentication of the user based at least in part on authenticating the identity of the user.

In some examples, the one or more characteristics of the environment of the user comprise a location of the user, an ambient light level, an ambient noise level, a voice of the user, or any combination thereof.

In some examples, the first wearable device comprises a wearable ring device, a wrist-worn wearable device, or both. In some examples, the second wearable device comprises wearable smart glasses.

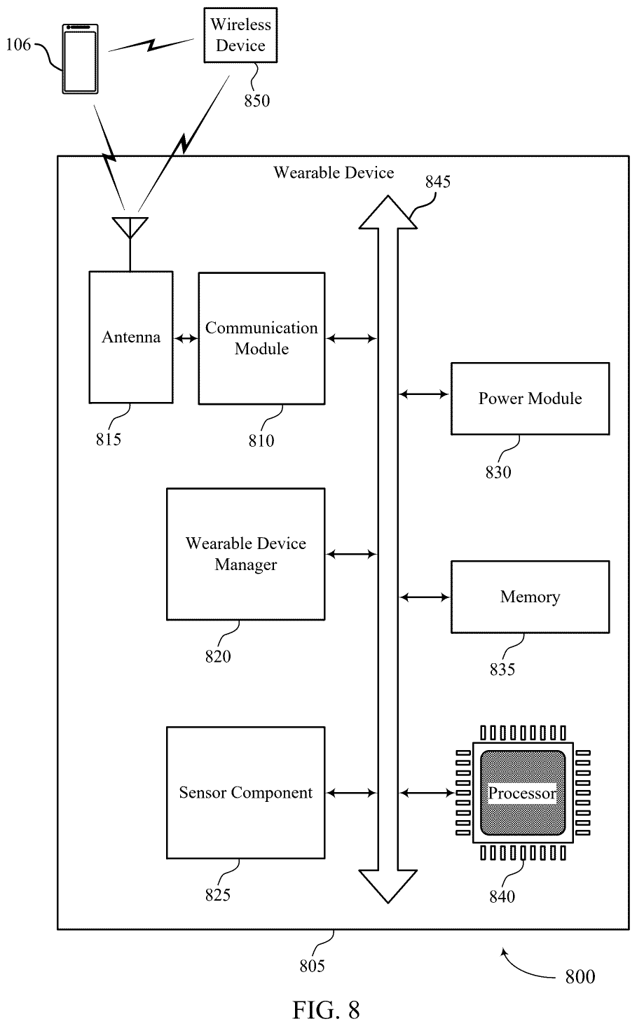

FIG. 8 shows a diagram of a system 800 including a device 805 that supports techniques for multiple wearable devices in accordance with aspects of the present disclosure. The device 805 may be an example of or include components of a device 605 as described herein. The device 805 may include an example of a wearable device 104, as described previously herein. The device 805 may include components for bi-directional communications including components for transmitting and receiving communications with a user device 106 and a server 110, such as a wearable device manager 820, a communication module 810, one or more antennas 815, a sensor component 825, a power module 830, at least one memory 835, at least one processor 840, and a wireless device 850. These components may be in electronic communication or otherwise coupled (e.g., operatively, communicatively, functionally, electronically, electrically) via one or more buses (e.g., a bus 845).

For example, the wearable device manager 820 may be configured as or otherwise support a means for acquiring physiological data from a user via a first wearable device worn at a first position on a body of the user, the physiological data comprising heart rate data, respiration rate data, motion data, or any combination thereof. The wearable device manager 820 may be configured as or otherwise support a means for acquiring environmental data associated with one or more characteristics of an environment of the user via a second wearable device worn at a second position on the body of the user, the environmental data comprising image data, audio data, or both. The wearable device manager 820 may be configured as or otherwise support a means for identifying one or more physiological characteristics of the user, one or more characteristics of the environment of the user, or both, based at least in part on the physiological data and the environmental data, respectively. The wearable device manager 820 may be configured as or otherwise support a means for causing the first wearable device to adjust one or more measurement characteristics used to acquire the physiological data based at least in part on the one or more characteristics of the environment of the user, or cause the second wearable device to acquire additional environmental data based at least in part on the one or more physiological characteristics of the user.

For example, the wearable device manager 820 may be configured as or otherwise support a means for acquiring physiological data from a user via a first wearable device worn at a first position on a body of the user, the physiological data comprising heart rate data, respiration rate data, motion data, or any combination thereof. The wearable device manager 820 may be configured as or otherwise support a means for acquiring environmental data associated with one or more characteristics of an environment of the user via a second wearable device worn at a second position on the body of the user, the environmental data comprising image data, audio data, or both. The wearable device manager 820 may be configured as or otherwise support a means for identifying one or more impacts that the environment of the user had on the physiological data of the user based at least in part on correlating a first portion of the physiological data collected during a time interval with a first portion of the image data, the audio data, or both, collected during the time interval. The wearable device manager 820 may be configured as or otherwise support a means for generating one or more signals configured to cause a user device, the second wearable device, or both, to provide a message comprising data associated with the one or more impacts.

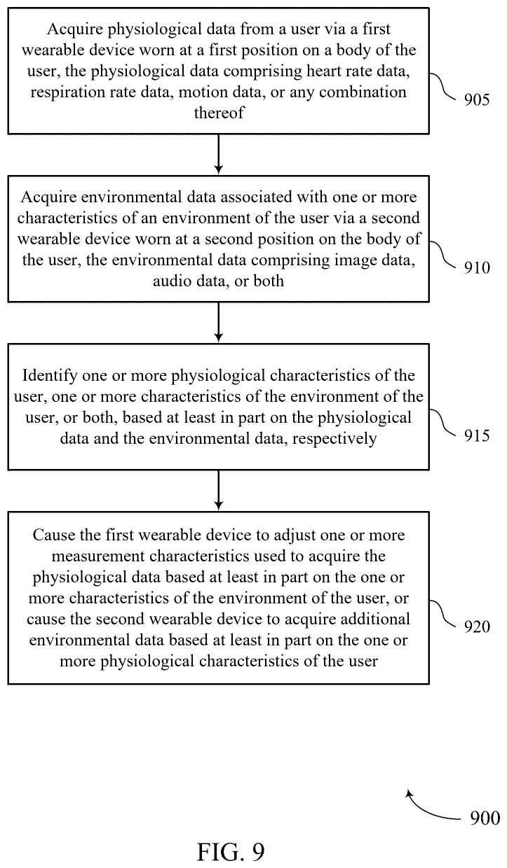

FIG. 9 shows a flowchart illustrating a method 900 that supports techniques for multiple wearable devices in accordance with aspects of the present disclosure. The operations of the method 900 may be implemented by a wearable device or its components as described herein. For example, the operations of the method 900 may be performed by a wearable device as described with reference to FIGS. 1 through 8. In some examples, a wearable device may execute a set of instructions to control the functional elements of the wearable device to perform the described functions. Additionally, or alternatively, the wearable device may perform aspects of the described functions using special-purpose hardware.

At 905, the method may include acquiring physiological data from a user via a first wearable device worn at a first position on a body of the user, the physiological data comprising heart rate data, respiration rate data, motion data, or any combination thereof. The operations of 905 may be performed in accordance with examples as disclosed herein. In some examples, aspects of the operations of 905 may be performed by a first wearable device manager 725 as described with reference to FIG. 7.

At 910, the method may include acquiring environmental data associated with one or more characteristics of an environment of the user via a second wearable device worn at a second position on the body of the user, the environmental data comprising image data, audio data, or both. The operations of 910 may be performed in accordance with examples as disclosed herein. In some examples, aspects of the operations of 910 may be performed by a second wearable device manager 730 as described with reference to FIG. 7.

At 915, the method may include identifying one or more physiological characteristics of the user, one or more characteristics of the environment of the user, or both, based at least in part on the physiological data and the environmental data, respectively. The operations of 915 may be performed in accordance with examples as disclosed herein. In some examples, aspects of the operations of 915 may be performed by a characteristic manager 735 as described with reference to FIG. 7.

At 920, the method may include causing the first wearable device to adjust one or more measurement characteristics used to acquire the physiological data based at least in part on the one or more characteristics of the environment of the user, or cause the second wearable device to acquire additional environmental data based at least in part on the one or more physiological characteristics of the user. The operations of 920 may be performed in accordance with examples as disclosed herein. In some examples, aspects of the operations of 920 may be performed by an output manager 740 as described with reference to FIG. 7.

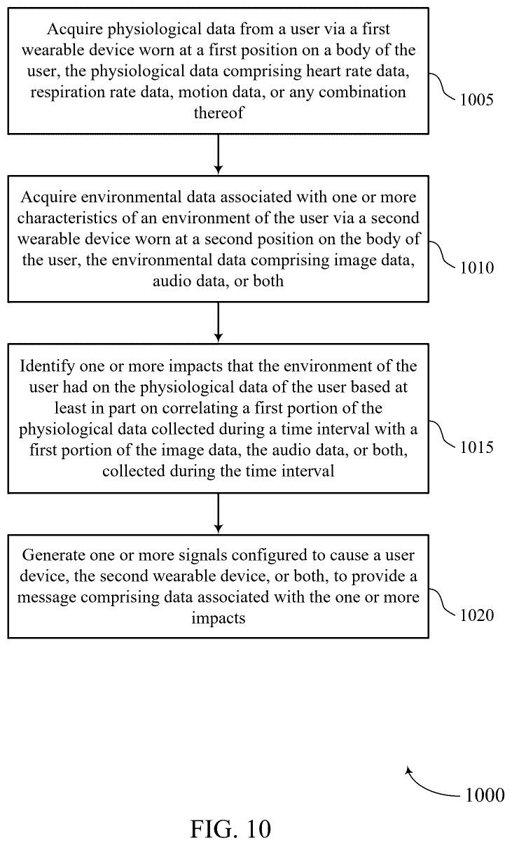

FIG. 10 shows a flowchart illustrating a method 1000 that supports techniques for multiple wearable devices in accordance with aspects of the present disclosure. The operations of the method 1000 may be implemented by a wearable device or its components as described herein. For example, the operations of the method 1000 may be performed by a wearable device as described with reference to FIGS. 1 through 8. In some examples, a wearable device may execute a set of instructions to control the functional elements of the wearable device to perform the described functions. Additionally, or alternatively, the wearable device may perform aspects of the described functions using special-purpose hardware.

At 1005, the method may include acquiring physiological data from a user via a first wearable device worn at a first position on a body of the user, the physiological data comprising heart rate data, respiration rate data, motion data, or any combination thereof. The operations of 1005 may be performed in accordance with examples as disclosed herein. In some examples, aspects of the operations of 1005 may be performed by a first wearable device manager 625 as described with reference to FIG. 6.

At 1010, the method may include acquiring environmental data associated with one or more characteristics of an environment of the user via a second wearable device worn at a second position on the body of the user, the environmental data comprising image data, audio data, or both. The operations of 1010 may be performed in accordance with examples as disclosed herein. In some examples, aspects of the operations of 1010 may be performed by a second wearable device manager 630 as described with reference to FIG. 6.

At 1015, the method may include identifying one or more impacts that the environment of the user had on the physiological data of the user based at least in part on correlating a first portion of the physiological data collected during a time interval with a first portion of the image data, the audio data, or both, collected during the time interval. The operations of 1015 may be performed in accordance with examples as disclosed herein. In some examples, aspects of the operations of 1015 may be performed by an environmental impact manager 645 as described with reference to FIG. 6.

At 1020, the method may include generating one or more signals configured to cause a user device, the second wearable device, or both, to provide a message comprising data associated with the one or more impacts. The operations of 1020 may be performed in accordance with examples as disclosed herein. In some examples, aspects of the operations of 1020 may be performed by an output module 615 as described with reference to FIG. 6.

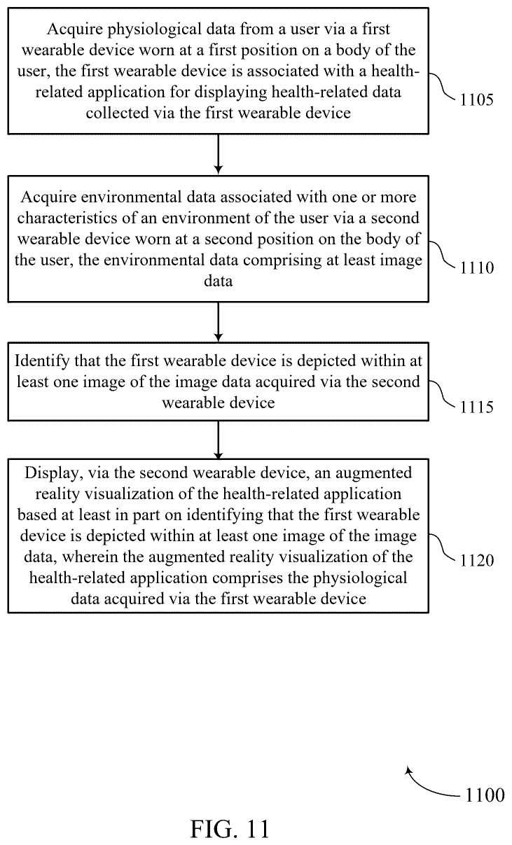

FIG. 11 shows a flowchart illustrating a method 1100 that supports techniques for multiple wearable devices in accordance with aspects of the present disclosure. The operations of the method 1100 may be implemented by a wearable device or its components as described herein. For example, the operations of the method 1100 may be performed by a wearable device as described with reference to FIGS. 1 through 8. In some examples, a wearable device may execute a set of instructions to control the functional elements of the wearable device to perform the described functions. Additionally, or alternatively, the wearable device may perform aspects of the described functions using special-purpose hardware.

At 1105, the method may include acquiring physiological data from a user via a first wearable device worn at a first position on a body of the user, the physiological data comprising heart rate data, respiration rate data, motion data, or any combination thereof. The operations of 1105 may be performed in accordance with examples as disclosed herein. In some examples, aspects of the operations of 1105 may be performed by a first wearable device manager 625 as described with reference to FIG. 6.

At 1110, the method may include acquiring environmental data associated with one or more characteristics of an environment of the user via a second wearable device worn at a second position on the body of the user, the environmental data comprising image data, audio data, or both. The operations of 1110 may be performed in accordance with examples as disclosed herein. In some examples, aspects of the operations of 1110 may be performed by a second wearable device manager 630 as described with reference to FIG. 6.

At 1115, the method may include identifying that the first wearable device is depicted within at least one image of the image data acquired via the second wearable device. The operations of 1115 may be performed in accordance with examples as disclosed herein. In some examples, aspects of the operations of 1115 may be performed by an environmental impact manager 645 as described with reference to FIG. 6.

At 1120, the method may include displaying, via the second wearable device, an augmented reality visualization of the health-related application based at least in part on identifying that the first wearable device is depicted within at least one image of the image data, wherein the augmented reality visualization of the health-related application comprises the physiological data acquired via the first wearable device. The operations of 1120 may be performed in accordance with examples as disclosed herein. In some examples, aspects of the operations of 1120 may be performed by an output module 615 as described with reference to FIG. 6.

It should be noted that the methods described above describe possible implementations, and that the operations and the steps may be rearranged or otherwise modified and that other implementations are possible. Furthermore, aspects from two or more of the methods may be combined.

A method by an apparatus is described. The method may include acquiring physiological data from a user via a first wearable device worn at a first position on a body of the user, the physiological data comprising heart rate data, respiration rate data, motion data, or any combination thereof, acquiring environmental data associated with one or more characteristics of an environment of the user via a second wearable device worn at a second position on the body of the user, the environmental data comprising image data, audio data, or both, identifying one or more physiological characteristics of the user, one or more characteristics of the environment of the user, or both, based at least in part on the physiological data and the environmental data, respectively, and causing the first wearable device to adjust one or more measurement characteristics used to acquire the physiological data based at least in part on the one or more characteristics of the environment of the user, or cause the second wearable device to acquire additional environmental data based at least in part on the one or more physiological characteristics of the user.

An apparatus is described. The apparatus may include one or more memories storing processor executable code, and one or more processors coupled with the one or more memories. The one or more processors may individually or collectively be operable to execute the code to cause the apparatus to acquire physiological data from a user via a first wearable device worn at a first position on a body of the user, the physiological data comprising heart rate data, respiration rate data, motion data, or any combination thereof, acquire environmental data associated with one or more characteristics of an environment of the user via a second wearable device worn at a second position on the body of the user, the environmental data comprising image data, audio data, or both, identify one or more physiological characteristics of the user, one or more characteristics of the environment of the user, or both, based at least in part on the physiological data and the environmental data, respectively, and cause the first wearable device to adjust one or more measurement characteristics used to acquire the physiological data based at least in part on the one or more characteristics of the environment of the user, or cause the second wearable device to acquire additional environmental data based at least in part on the one or more physiological characteristics of the user.

Another apparatus is described. The apparatus may include means for acquiring physiological data from a user via a first wearable device worn at a first position on a body of the user, the physiological data comprising heart rate data, respiration rate data, motion data, or any combination thereof, means for acquiring environmental data associated with one or more characteristics of an environment of the user via a second wearable device worn at a second position on the body of the user, the environmental data comprising image data, audio data, or both, means for identifying one or more physiological characteristics of the user, one or more characteristics of the environment of the user, or both, based at least in part on the physiological data and the environmental data, respectively, and means for causing the first wearable device to adjust one or more measurement characteristics used to acquire the physiological data based at least in part on the one or more characteristics of the environment of the user, or cause the second wearable device to acquire additional environmental data based at least in part on the one or more physiological characteristics of the user.

A non-transitory computer-readable medium storing code is described. The code may include instructions executable by one or more processors to acquire physiological data from a user via a first wearable device worn at a first position on a body of the user, the physiological data comprising heart rate data, respiration rate data, motion data, or any combination thereof, acquire environmental data associated with one or more characteristics of an environment of the user via a second wearable device worn at a second position on the body of the user, the environmental data comprising image data, audio data, or both, identify one or more physiological characteristics of the user, one or more characteristics of the environment of the user, or both, based at least in part on the physiological data and the environmental data, respectively, and cause the first wearable device to adjust one or more measurement characteristics used to acquire the physiological data based at least in part on the one or more characteristics of the environment of the user, or cause the second wearable device to acquire additional environmental data based at least in part on the one or more physiological characteristics of the user.

Some examples of the method, apparatus, and non-transitory computer-readable medium described herein may further include operations, features, means, or instructions for identifying that the user may be engaged in a physical activity based at least in part on the physiological data collected via the first wearable device and determining an activity type of the physical activity based at least in part on the physiological data collected via the first wearable device and the environmental data acquired via the second wearable device.

Some examples of the method, apparatus, and non-transitory computer-readable medium described herein may further include operations, features, means, or instructions for identifying a gesture performed by the user based at least in part on the motion data collected via the first wearable device and causing the second wearable device to save a portion of the image data, save a portion of the audio data, collect additional image data, collect additional audio data, or any combination thereof, based at least in part on identifying the gesture.

Some examples of the method, apparatus, and non-transitory computer-readable medium described herein may further include operations, features, means, or instructions for receiving, from an external device, a request to authenticate the user, authenticating an identity of the user based at least in part on the physiological data collected via the first wearable device and the environmental data collected via the second wearable device, and outputting a message to the external device comprising an authentication of the user based at least in part on authenticating the identity of the user.

In some examples of the method, apparatus, and non-transitory computer-readable medium described herein, the one or more characteristics of the environment of the user comprise a location of the user, an ambient light level, an ambient noise level, a voice of the user, or any combination thereof.

In some examples of the method, apparatus, and non-transitory computer-readable medium described herein, the first wearable device comprises a wearable ring device, a wrist-worn wearable device, or both and the second wearable device comprises wearable smart glasses.

A method by an apparatus is described. The method may include acquiring physiological data from a user via a first wearable device worn at a first position on a body of the user, the physiological data comprising heart rate data, respiration rate data, motion data, or any combination thereof, acquiring environmental data associated with one or more characteristics of an environment of the user via a second wearable device worn at a second position on the body of the user, the environmental data comprising image data, audio data, or both, identifying one or more impacts that the environment of the user had on the physiological data of the user based at least in part on correlating a first portion of the physiological data collected during a time interval with a first portion of the image data, the audio data, or both, collected during the time interval, and generating one or more signals configured to cause a user device, the second wearable device, or both, to provide a message comprising data associated with the one or more impacts.

An apparatus is described. The apparatus may include one or more memories storing processor executable code, and one or more processors coupled with the one or more memories. The one or more processors may individually or collectively be operable to execute the code to cause the apparatus to acquire physiological data from a user via a first wearable device worn at a first position on a body of the user, the physiological data comprising heart rate data, respiration rate data, motion data, or any combination thereof, acquire environmental data associated with one or more characteristics of an environment of the user via a second wearable device worn at a second position on the body of the user, the environmental data comprising image data, audio data, or both, identify one or more impacts that the environment of the user had on the physiological data of the user based at least in part on correlating a first portion of the physiological data collected during a time interval with a first portion of the image data, the audio data, or both, collected during the time interval, and generate one or more signals configured to cause a user device, the second wearable device, or both, to provide a message comprising data associated with the one or more impacts.

Another apparatus is described. The apparatus may include means for acquiring physiological data from a user via a first wearable device worn at a first position on a body of the user, the physiological data comprising heart rate data, respiration rate data, motion data, or any combination thereof, means for acquiring environmental data associated with one or more characteristics of an environment of the user via a second wearable device worn at a second position on the body of the user, the environmental data comprising image data, audio data, or both, means for identifying one or more impacts that the environment of the user had on the physiological data of the user based at least in part on correlating a first portion of the physiological data collected during a time interval with a first portion of the image data, the audio data, or both, collected during the time interval, and means for generating one or more signals configured to cause a user device, the second wearable device, or both, to provide a message comprising data associated with the one or more impacts.

A non-transitory computer-readable medium storing code is described. The code may include instructions executable by one or more processors to acquire physiological data from a user via a first wearable device worn at a first position on a body of the user, the physiological data comprising heart rate data, respiration rate data, motion data, or any combination thereof, acquire environmental data associated with one or more characteristics of an environment of the user via a second wearable device worn at a second position on the body of the user, the environmental data comprising image data, audio data, or both, identify one or more impacts that the environment of the user had on the physiological data of the user based at least in part on correlating a first portion of the physiological data collected during a time interval with a first portion of the image data, the audio data, or both, collected during the time interval, and generate one or more signals configured to cause a user device, the second wearable device, or both, to provide a message comprising data associated with the one or more impacts.

Some examples of the method, apparatus, and non-transitory computer-readable medium described herein may further include operations, features, means, or instructions for identifying one or more characteristics of the environment of the user based at least in part on the image data, the audio data, or both, collected via the second wearable device and causing the first wearable device to adjust one or more measurement characteristics used to acquire the physiological data based at least in part on the one or more characteristics of the environment of the user, wherein the first wearable device may be configured to acquire additional physiological data based at least in part on adjusting the one or more measurement characteristics.

Some examples of the method, apparatus, and non-transitory computer-readable medium described herein may further include operations, features, means, or instructions for identifying one or more physiological characteristics of the user based at least in part on the image data, the audio data, or both, collected via the second wearable device and causing the second wearable device to acquire additional image data, additional audio data, or both, based at least in part on the one or more physiological characteristics of the user.

Some examples of the method, apparatus, and non-transitory computer-readable medium described herein may further include operations, features, means, or instructions for identifying that the user may be engaged in a physical activity based at least in part on the physiological data collected via the first wearable device and determining an activity type of the physical activity based at least in part on the physiological data collected via the first wearable device and the environmental data acquired via the second wearable device.

Some examples of the method, apparatus, and non-transitory computer-readable medium described herein may further include operations, features, means, or instructions for identifying a gesture performed by the user based at least in part on the motion data collected via the first wearable device and causing the second wearable device to save a portion of the image data, save a portion of the audio data, collect additional image data, collect additional audio data, or any combination thereof, based at least in part on identifying the gesture.

Some examples of the method, apparatus, and non-transitory computer-readable medium described herein may further include operations, features, means, or instructions for receiving, from an external device, a request to authenticate the user, authenticating an identity of the user based at least in part on the physiological data collected via the first wearable device and the environmental data collected via the second wearable device, and outputting a message to the external device comprising an authentication of the user based at least in part on authenticating the identity of the user.

In some examples of the method, apparatus, and non-transitory computer-readable medium described herein, the one or more characteristics of the environment of the user comprise a location of the user, an ambient light level, an ambient noise level, a voice of the user, or any combination thereof.

In some examples of the method, apparatus, and non-transitory computer-readable medium described herein, the first wearable device comprises a wearable ring device, a wrist-worn wearable device, or both and the second wearable device comprises wearable smart glasses.

A method by an apparatus is described. The method may include acquiring image data associated with an environment of a user via one or more image capture devices of a smart glasses device worn by the user, identifying, via one or more processors of the smart glasses device, that a wearable device worn by the user is depicted within at least one image of the image data acquired via the one or more image capture devices, the wearable device configured to acquire physiological data from the user, wherein the wearable device is associated with a health-related application for displaying health-related data collected via the wearable device, accessing, via the one or more processors, the health-related application associated with the wearable device based at least in part on identifying that the wearable device is depicted within the at least one image of the image data, and causing a display device of the smart glasses device to display an augmented reality visualization of the health-related application via one or more lenses of the smart glasses device based at least in part on identifying that the wearable device is depicted within at least one image of the image data and based at least in part on accessing the health-related application.

An apparatus is described. The apparatus may include one or more memories storing processor executable code, and one or more processors coupled with the one or more memories. The one or more processors may individually or collectively be operable to execute the code to cause the apparatus to acquire image data associated with an environment of a user via one or more image capture devices of a smart glasses device worn by the user, identify, via one or more processors of the smart glasses device, that a wearable device worn by the user is depicted within at least one image of the image data acquired via the one or more image capture devices, the wearable device configured to acquire physiological data from the user, wherein the wearable device is associated with a health-related application for displaying health-related data collected via the wearable device, access, via the one or more processors, the health-related application associated with the wearable device based at least in part on identifying that the wearable device is depicted within the at least one image of the image data, and cause a display device of the smart glasses device to display an augmented reality visualization of the health-related application via one or more lenses of the smart glasses device based at least in part on identifying that the wearable device is depicted within at least one image of the image data and based at least in part on accessing the health-related application.

Another apparatus is described. The apparatus may include means for acquiring image data associated with an environment of a user via one or more image capture devices of a smart glasses device worn by the user, means for identifying, via one or more processors of the smart glasses device, that a wearable device worn by the user is depicted within at least one image of the image data acquired via the one or more image capture devices, the wearable device configured to acquire physiological data from the user, wherein the wearable device is associated with a health-related application for displaying health-related data collected via the wearable device, means for accessing, via the one or more processors, the health-related application associated with the wearable device based at least in part on identifying that the wearable device is depicted within the at least one image of the image data, and means for causing a display device of the smart glasses device to display an augmented reality visualization of the health-related application via one or more lenses of the smart glasses device based at least in part on identifying that the wearable device is depicted within at least one image of the image data and based at least in part on accessing the health-related application.

A non-transitory computer-readable medium storing code is described. The code may include instructions executable by one or more processors to acquire image data associated with an environment of a user via one or more image capture devices of a smart glasses device worn by the user, identify, via one or more processors of the smart glasses device, that a wearable device worn by the user is depicted within at least one image of the image data acquired via the one or more image capture devices, the wearable device configured to acquire physiological data from the user, wherein the wearable device is associated with a health-related application for displaying health-related data collected via the wearable device, access, via the one or more processors, the health-related application associated with the wearable device based at least in part on identifying that the wearable device is depicted within the at least one image of the image data, and cause a display device of the smart glasses device to display an augmented reality visualization of the health-related application via one or more lenses of the smart glasses device based at least in part on identifying that the wearable device is depicted within at least one image of the image data and based at least in part on accessing the health-related application.

In some examples of the method, apparatus, and non-transitory computer-readable medium described herein, the augmented reality visualization comprises a visual depiction of the physiological data acquired via the wearable device.

A method by an apparatus is described. The method may include transmitting a signal from the smart glasses device to the wearable device based at least in part on identifying that the wearable device is depicted within the at least one image of the image data, wherein the signal is configured to cause the wearable device to perform one or more measurements and receiving, in response to the signal, information associated with the one or more measurements performed by the wearable device, wherein the augmented reality visualization comprises a visual depiction of the one or more measurements.

An apparatus is described. The apparatus may include one or more memories storing processor executable code, and one or more processors coupled with the one or more memories. The one or more processors may individually or collectively be operable to execute the code to cause the apparatus to transmit a signal from the smart glasses device to the wearable device based at least in part on identifying that the wearable device is depicted within the at least one image of the image data, wherein the signal is configured to cause the wearable device to perform one or more measurements and receive, in response to the signal, information associated with the one or more measurements performed by the wearable device, wherein the augmented reality visualization comprises a visual depiction of the one or more measurements.

Another apparatus is described. The apparatus may include means for transmitting a signal from the smart glasses device to the wearable device based at least in part on identifying that the wearable device is depicted within the at least one image of the image data, wherein the signal is configured to cause the wearable device to perform one or more measurements and means for receiving, in response to the signal, information associated with the one or more measurements performed by the wearable device, wherein the augmented reality visualization comprises a visual depiction of the one or more measurements.

A non-transitory computer-readable medium storing code is described. The code may include instructions executable by one or more processors to transmit a signal from the smart glasses device to the wearable device based at least in part on identifying that the wearable device is depicted within the at least one image of the image data, wherein the signal is configured to cause the wearable device to perform one or more measurements and receive, in response to the signal, information associated with the one or more measurements performed by the wearable device, wherein the augmented reality visualization comprises a visual depiction of the one or more measurements.

In some examples of the method, apparatus, and non-transitory computer-readable medium described herein, the one or more measurements are received in real-time or near-real time and the augmented reality visualization displays the visual depiction of the one or more measurements in real-time or near-real time.

An apparatus is described. The apparatus may include one or more memories storing processor executable code, and one or more processors coupled with the one or more memories. The one or more processors may individually or collectively be operable to execute the code to cause the apparatus to receive, via the one or more processors of the smart glasses device, the physiological data collected via the wearable device, determine that the physiological data satisfies one or more trigger conditions, and capture additional image data via the one or more image capture devices based at least in part on the physiological data satisfying the one or more trigger conditions.

Another apparatus is described. The apparatus may include means for receiving, via the one or more processors of the smart glasses device, the physiological data collected via the wearable device, means for determining that the physiological data satisfies one or more trigger conditions, and means for capturing additional image data via the one or more image capture devices based at least in part on the physiological data satisfying the one or more trigger conditions.

A non-transitory computer-readable medium storing code is described. The code may include instructions executable by one or more processors to receive, via the one or more processors of the smart glasses device, the physiological data collected via the wearable device, determine that the physiological data satisfies one or more trigger conditions, and capture additional image data via the one or more image capture devices based at least in part on the physiological data satisfying the one or more trigger conditions.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A smart glasses device configured to be worn on a head of a user, the smart glasses device comprising:
one or more lenses;
a display device configured to display visual information to the user via the one or more lenses;
one or more image capture devices configured to capture image data associated with an environment of the user; and
one or more processors electrically coupled with the display device and the one or more image capture devices, wherein the one or more processors are individually or collectively configured to cause the smart glasses device to:
identify that a wearable device worn by the user is depicted within at least one image of the image data acquired via the one or more image capture devices, the wearable device configured to acquire physiological data from the user, wherein the wearable device is associated with a health-related application for displaying health-related data collected via the wearable device; and
display, via the display device, an augmented reality visualization of the health-related application based at least in part on identifying that the wearable device is depicted within the at least one image of the image data.

2. The smart glasses device of claim 1, wherein the augmented reality visualization comprises a visual depiction of the physiological data acquired via the wearable device.

3. The smart glasses device of claim 1, wherein the one or more processors are individually or collectively configured to cause the smart glasses device to:
access the health-related application via a wireless connection with a user device associated with the wearable device based at least in part on identifying that the wearable device is depicted within the at least one image of the image data, wherein the health-related application is executable on the user device, and wherein the augmented reality visualization of the health-related application is displayed based at least in part on accessing the health-related application via the wireless connection with the user device.

4. The smart glasses device of claim 1, wherein the one or more processors are individually or collectively configured to cause the smart glasses device to:
transmit a signal to the wearable device based at least in part on identifying that the wearable device is depicted within the at least one image of the image data, wherein the signal is configured to cause the wearable device to perform one or more measurements; and
receive, in response to the signal, information associated with the one or more measurements performed by the wearable device, wherein the augmented reality visualization comprises a visual depiction of the one or more measurements.

5. The smart glasses device of claim 4, wherein the information associated with the one or more measurements is received in real-time or near-real time, and wherein the augmented reality visualization displays the visual depiction of the one or more measurements in real-time or near-real time.

6. The smart glasses device of claim 1, wherein the one or more processors are individually or collectively configured to cause the smart glasses device to:
identify a gesture performed by the user based at least in part on the image data acquired via the one or more image capture devices, the physiological data acquired via the wearable device, or both, wherein the augmented reality visualization of the health-related application is displayed based at least in part on identification of the gesture.

7. The smart glasses device of claim 1, wherein the one or more processors are individually or collectively configured to cause the smart glasses device to:
identify a gesture performed by the user based at least in part on the image data acquired via the one or more image capture devices, the physiological data acquired via the wearable device, or both; and
selectively adjust or control the augmented reality visualization of the health-related application based at least in part on identification of the gesture.

8. The smart glasses device of claim 1, wherein the one or more processors are individually or collectively configured to cause the smart glasses device to:
receive the physiological data collected via the wearable device;
determine that the physiological data satisfies one or more trigger conditions; and
capture additional image data via the one or more image capture devices based at least in part on the physiological data satisfying the one or more trigger conditions.

9. The smart glasses device of claim 8, further comprising one or more audio capture devices, wherein the one or more processors are individually or collectively configured to cause the smart glasses device to:
capture audio data associated with the environment of the user using the one or more audio capture devices based at least in part on the physiological data satisfying the one or more trigger conditions.

10. The smart glasses device of claim 1, wherein the one or more processors are individually or collectively configured to cause the smart glasses device to:
authenticate an identity of the user based at least in part on the physiological data collected via the wearable device, and based at least in part on the image data acquired via the one or more image capture devices, audio data acquired via one or more audio capture devices of the smart glasses device, or both; and
transmit a signal to an external device comprising an authentication of the user based at least in part on authenticating the identity of the user, wherein the signal is configured to cause the external device to perform one or more actions.

11. A method, comprising:
acquiring image data associated with an environment of a user via one or more image capture devices of a smart glasses device worn by the user;
identifying, via one or more processors of the smart glasses device, that a wearable device worn by the user is depicted within at least one image of the image data acquired via the one or more image capture devices, the wearable device configured to acquire physiological data from the user, wherein the wearable device is associated with a health-related application for displaying health-related data collected via the wearable device;
accessing, via the one or more processors, the health-related application associated with the wearable device based at least in part on identifying that the wearable device is depicted within the at least one image of the image data; and
causing a display device of the smart glasses device to display an augmented reality visualization of the health-related application via one or more lenses of the smart glasses device based at least in part on identifying that the wearable device is depicted within the at least one image of the image data and based at least in part on accessing the health-related application.

12. The method of claim 11, wherein the augmented reality visualization comprises a visual depiction of the physiological data acquired via the wearable device.

13. The method of claim 11, further comprising:
transmitting a signal from the smart glasses device to the wearable device based at least in part on identifying that the wearable device is depicted within the at least one image of the image data, wherein the signal is configured to cause the wearable device to perform one or more measurements; and
receiving, in response to the signal, information associated with the one or more measurements performed by the wearable device, wherein the augmented reality visualization comprises a visual depiction of the one or more measurements.

14. The method of claim 13, wherein the information associated with the one or more measurements is received in real-time or near-real time, and wherein the augmented reality visualization displays the visual depiction of the one or more measurements in real-time or near-real time.

15. The method of claim 11, further comprising:
identifying a gesture performed by the user based at least in part on the image data acquired via the one or more image capture devices, the physiological data acquired via the wearable device, or both, wherein the augmented reality visualization of the health-related application is displayed based at least in part on identification of the gesture.

16. The method of claim 11, further comprising:
receiving, via the one or more processors of the smart glasses device, the physiological data collected via the wearable device;
determining that the physiological data satisfies one or more trigger conditions; and
capturing additional image data via the one or more image capture devices based at least in part on the physiological data satisfying the one or more trigger conditions.

17. A wearable system, comprising:
a first wearable device configured to acquire physiological data from a user, the physiological data comprising heart rate data, respiration rate data, motion data, or any combination thereof, the first wearable device worn at a first position on a body of the user;
a smart glasses device configured to be worn on a head of the user, the smart glasses device configured to acquire environmental data associated with one or more characteristics of an environment of the user, the environmental data comprising image data, audio data, or both; and
one or more processors communicatively coupled with the first wearable device, the smart glasses device, or both, wherein the one or more processors are configured to:
acquire the physiological data collected via the first wearable device and the environmental data collected via the smart glasses device;
identify one or more physiological characteristics of the user, one or more characteristics of the environment of the user, or both, based at least in part on the physiological data and the environmental data, respectively; and
cause the first wearable device to adjust one or more measurement characteristics used to acquire the physiological data based at least in part on the one or more characteristics of the environment of the user, or cause the smart glasses device to acquire additional environmental data based at least in part on the one or more physiological characteristics of the user.

18. The wearable system of claim 17, wherein the one or more processors are further configured to:
identify that the user is engaged in a physical activity based at least in part on the physiological data collected via the first wearable device; and
determine an activity type of the physical activity based at least in part on the physiological data collected via the first wearable device and the environmental data acquired via the smart glasses device.

19. The wearable system of claim 17, wherein the one or more processors are further configured to:
identify a gesture performed by the user based at least in part on the motion data collected via the first wearable device; and
cause the smart glasses device to save a portion of the image data, save a portion of the audio data, collect additional image data, collect additional audio data, or any combination thereof, based at least in part on identifying the gesture.

20. The wearable system of claim 17, wherein the one or more processors are further configured to:
receive, from an external device, a request to authenticate the user;
authenticate an identity of the user based at least in part on the physiological data collected via the first wearable device and the environmental data collected via the smart glasses device; and
output a message to the external device comprising an authentication of the user based at least in part on authenticating the identity of the user.
